(19) United States
(12) Patent Application Publication
(10) Pub. No.: US 2005/0192497 A1
(43) Pub. Date: Sep. 1, 2005

(54) Magnetic resonance imaging method

(76) Inventors: Jeffrey Tsao, Basel (CH); Klaas Paul Pruessmann, Zurich (CH); Peter Boesiger, Ennebaden (CH)

Correspondence Address: PHILIPS INTELLECTUAL PROPERTY & STANDARDS, 595 MINER ROAD, CLEVELAND, OH, 44143, US

(21) Appl. No.: 10/514,326

(22) PCT Filed: May 12, 2003
(86) PCT No.: PCT/IB03/01988

§ 371 Date: Nov. 12, 2004

(30) Foreign Application Priority Data
May 13, 2002 (EP) 02076838.8

(51) Int. Cl. A61B 5/05

(52) U.S. Cl. 600/421

(57) ABSTRACT

In SENSitivity Encoding (SENSE), the reconstructed images are susceptible to amplified noise and/or artifacts if the underlying matrix inversion procedure is ill-conditioned. In this work, we propose to firstly apply the conventional SENSE algorithm to obtain an initial estimate. This initial estimate undergoes filtering to improve the signal-to-noise ratio. Then, it is fed back to the reconstruction as a reference image to estimate the amount of aliasing that may arise from regularization. We derive the optimal regularized solution that minimizes the weighted sum of artifact and noise power.

The present invention relates to a magnetic resonance imaging method wherein undersampled magnetic resonance signals are acquired by a receiver antennae system having a spatial sensitivity profile and the image being reconstructed from the undersampled magnetic resonance signals and the spatial sensitivity profile.

In the method of undersampled acquisition known as SENSitivity Encoding (SENSE), as described by Pruessmann K P et al. in MRM 1999; 42:952-962, the reconstructed images are susceptible to amplified noise and/or artifacts if the underlying matrix inversion procedure is ill-conditioned.

It is therefore an object of the present invention to improve the above mentioned method of undersampled acquisition by reducing the amount of noise in the final image in order to handle scenarios with a low signal-to-noise ratio (SNR) and/or a high reduction factor R.

This object is achieved by the magnetic resonance imaging method according to claim 1, whereas the reconstruction of the image is provided by a first step, in which image is reconstructed on the basis of reconstruction matrices according to a parallel imaging like SENSE, thereinafter the so reconstructed image is subject to a filtering operation, which provides a post-processed image, which is used to alter the reconstruction matrices, and by a second step, in which the final image is reconstructed on the basis of the altered reconstruction matrices. Using this scheme, the reconstruction from the second step is optimized with respect to minimizing noise and aliasing artifacts. The further objects are achieved by the magnetic resonance imaging system according to claim 8 and the computer program product according to claim 9.

The method according to the present invention has the advantage, that the amount of noise artifacts in the image can be reduced without any influence on the sampling rate, i.e. the reduction factor R.

These and other aspects of the invention will be elaborated with reference to the preferred implementations as defined in the dependent claims. In the following description an exemplified embodiment of the invention is described with respect to the accompanying drawings. It shows

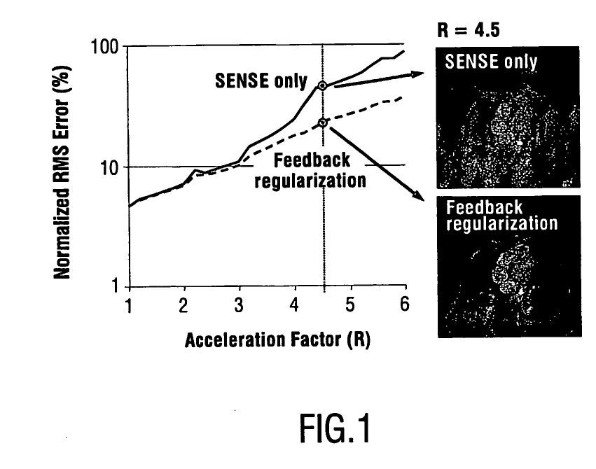

FIG. 1 a diagram of the acceleration factor R versus the normalized RMS error (left) and an reconstructed image with SENSE only and with feedback regularization (right), and

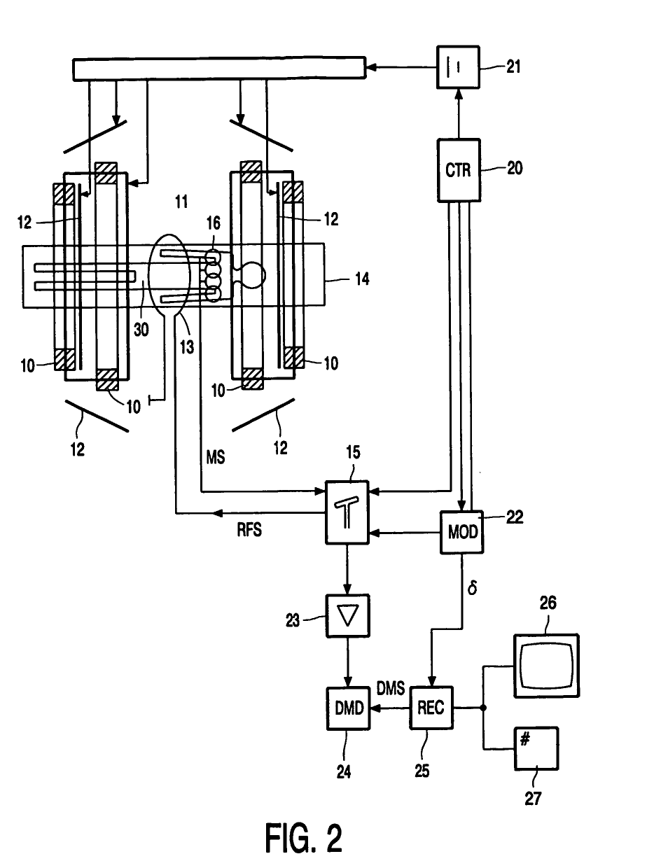

FIG. 2 diagrammatically a magnetic resonance imaging system in which the invention is used.

In the present description a multiple of receiver antenna or coils are used. However, it is also possible to implement the SENSE method with a single receiving coil or antenna at different receiving positions.

Basic Principles

At the heart of the SENSitivity Encoding (SENSE), a parallel imaging (PI) method, as described in Pruessmann K P, et al. Magn Reson Med 42:952-962, 1999, lies a series of matrix inversions that determine the unaliased image voxels v from the measured k-space data a This linear system can be represented as:
v=(L⁻¹ S)⁺L⁻¹ a=F a [1]
where S denotes the so-called sensitivity matrix (1). L is the “square root” (e.g. by Cholesky decomposition) of the noise correlation matrix Ψ (2) (i.e. Ψ=L L^{H}). Superscript+denotes a (regularized) pseudo-inverse. Eq. [1] is equivalent to the original SENSE formulation as described in Pruessmann K P, et al. Magn Reson Med 42:952-962, 1999 and in Pruessmann K P, et al. Magn Reson Med 46:638-651, 2001.

If the matrix product (L⁻¹ S) is ill-conditioned, v is sensitive to perturbations (cf. Golub G H, Van Loan C F. Matrix computations. 3^{rd} ed. Johns Hopkins University Press, 1996) on the right hand side of Eq. [1], including measurement noise and inaccuracy of the sensitivity maps. A wide variety of regularization approaches exist to improve the conditioning of the inversion procedure (see e.g. Hansen P C. Numerical Algorithms 6:1-35, 1994). In all cases, accuracy of the inversion is traded off to gain stability. In the present work, we propose a two-pass procedure to estimate this trade-off quantitatively.

### PRACTICAL EXAMPLE OF THE INVENTION

In the first pass of the proposed method, the conventional SENSE algorithm is applied using only truncated singular value decomposition (SVD) to avoid obvious noise amplification (cutoff at condition number >100). This generates an initial estimate \hat{v}, which undergoes median filtering to improve the signal-to-noise ratio. In the second pass, the regularized reconstruction matrix F is determined as the solution that minimizes the following weighted sum:
α(Noise Power)+(Artifact Power)=α∥F L∥_{Frob}²+∥(F S−I)diag(\hat{v})∥_{Frob}² [2]
where α denote the weight given to the noise term relative to the artifact term (arbitrarily set to 1 in this work); ∥•∥_{Frob} denotes the Frobenius norm. The first term of Eq. [1] estimates the noise power of the reconstructed voxels, while the second term estimates the artifact power resulting from regularization assuming that the true voxel intensities are given by \hat{v}. Regardless of the regularization strategy used (e.g. diagonal loading, truncated SVD, damped SVD, etc.) the optimal reconstruction matrix Fₒₚₜ that minimizes Eq. [2] can be determined analytically, and it has several mathematically equivalent forms, including:

$$\begin{matrix}{F}_{opt} = {( {S}^{H} {ψ}^{- 1} S + α {diag ( {\vert \hat{v} \vert}^{2} )}^{- 1} )}^{- 1} {S}^{H} {ψ}^{- 1} \\ = diag ( {\vert \hat{v} \vert}^{2} ) {{S}^{H} ( S diag ( {\vert \hat{v} \vert}^{2} ) {S}^{H} + α ψ )}^{- 1}\end{matrix} \qquad [ 3 a , b ]$$

For α=1, these expressions become equivalent to those previously derived (8-9). An interesting observation is that the matrix product S diag(\hat{v}) is equal to the sensitivity maps multiplied by image estimate \hat{v}. This has been referred to as the “in vivo sensitivities” (cf. Wang J et al. Workshop on Parallel MR Imaging Basics and Clinical Applications. 89, 2001, and Sodickson D K. Magn Reson Med. 44:243-251, 2000). Thus, Eq. [3b] can be rewritten as follows, with S_{in vivo}=S diag(\hat{v}):
Fₒₚₜ=diag(\hat{v})S_{in vivo}^{H}(S_{in vivo}S_{in vivo}^{H}+α↓)⁻¹ [4]

In principle, the use of in vivo sensitivities has no effect on the reconstruction. In practice however, the in vivo sensitivities are typically acquired using the center of k-space (compare McKenzie C A, et al. Workshop on Parallel MR Imaging Basics and Clinical Applications. 88, 2001) or a separate low-resolution reference. Thus, the in vivo sensitivities are convolved with a low-pass point spread function. This approximation can be regarded as a modeling error in Eq. [1]. The maximum error amplification is bounded by the condition number of Fₒₚₜ (see Golub G H, Van Loan C F. Matrix computations. 3 ed. Baltimore: Johns Hopkins University Press, 1996.); while the minimum error is bounded by the reconstruction error from actually using accurate high-resolution in vivo sensitivities as S_{in vivo}.

Simulations were performed using a cardiac image (see e.g. Weiger M, et al. Magn Reson Med 43:177-184, 2000), with a six-element coil array placed around the body (see e.g. Weiger M, et al. Magn Reson Med 45:495-504, 2001), and a signal-to-noise ratio of 10. Root-mean-square (RMS) reconstruction error was determined as a function of the acceleration factor (R) along the phase-encoding direction (left-right).

FIG. 1 shows that the RMS error improves at all acceleration factors with regularization, including a marginal improvement even at R=1. This improvement at R=1 is due to the feedback mechanism serving as a a self-consistency check. In general, the amount of improvement strongly depends on the image contents, with larger improvements possible if the aliased voxels exhibit high contrasts. On the other hand, if the entire field-of-view has approximately uniform intensity, the improvement is negligible, as would be expected. The inset images show the reconstruction results with and without feedback regularization at R=4.5.

### CONCLUSION

In the present invention, we present a feedback framework for regularized reconstruction. We exploit the fact that neighboring voxels are highly correlated. As a result, filtering applied to the first-pass reconstruction can be used to obtain a high signal-to-noise image estimate, which can be used to estimate the potential amount of artifacts. In the case of dynamic imaging, temporal correlations (as described in Wang J et al. Workshop on Parallel MR Imaging Basics and Clinical Applications. 89, 2001) or joint spatiotemporal correlations may also be used to obtain the image estimate. For a given estimate, we applied median filtering to improve the image quality, but a wide variety of other filtering methods can be used as well, including anisotropic diffusion (see Gerig G, et al. IEEE Trans Med Imaging 11:221-232, 1992) and statistical approaches. Finally, the noise-versus-artifact tradeoff can also be evaluated in a number of manners (see e.g. Hansen P C. Numerical Algorithms 6:1-35, 1994). However, for any regularized reconstruction that minimizes the expression in Eq. [2], the reconstruction formula in Eqs. [3a, 3b, 4] represent the optimal, regardless of the regularization strategy used.

In the above presented method a number of filtering methods can be used, such as low-pass filtering, median filtering, statistical filtering, anisotropic filtering and wavelet filtering. Low-pass filtering involves blurring each voxel with its neighbours. Median filtering involves replacing the intensity of each voxel wiht the median of the voxel intensities within a neighbourhood. Statistical filtering involves comparing the statistical properties of each voxel to those of noise, and discarding or attenuating those voxels that are similar to noise. Anisotropic filtering involves blurring each voxel with its neighbours with the degree of blurring dependent on the degree of similarity between them. Wavelet filtering involves transforming an image from geometric space to wavelet space, which is spanned by a family of wavelet functions. The filtering is then applied in wavelet space using any of the above filtering methods. The filtered data are inverse-transformed back to geometric space.

FIG. 3 shows diagrammatically a magnetic resonance imaging System in which the invention is used.

The magnetic resonance imaging system includes a set of main coils 10 whereby a steady, uniform magnetic field is generated. The main coils are constructed, for example in such a manner that they enclose a tunnel-shaped examination space. The patient to be examined is slid on a table into this tunnel-shaped examination space. The magnetic resonance imaging system also includes a number of gradient coils 11, 12 whereby magnetic fields exhibiting spatial variations, notably in the form of temporary gradients in individual directions, are generated so as to be superposed on the uniform magnetic field. The gradient coils 11, 12 are connected to a controllable power supply unit 21. The gradient coils 11, 12 are energized by application of an electric current by means of the power supply unit 21. The strength, direction and duration of the gradients are controlled by control of the power supply unit. The magnetic resonance imaging system also includes transmission and receiving coils 13, 15 for generating RF excitation pulses and for picking up the magnetic resonance signals, respectively. The transmission coil 13 is preferably constructed as a body coil whereby (a part of) the object to be examined can be enclosed. The body coil is usually arranged in the magnetic resonance imaging system in such a manner that the patient 30 to be examined, being arranged in the magnetic resonance imaging system, is enclosed by the body coil 13. The body coil 13 acts as a transmission aerial for the transmission of the RF excitation pulses and RF refocusing pulses. Preferably, the body coil 13 involves a spatially uniform intensity distribution of the transmitted RF pulses. The receiving coils 15 are preferably surface coils 15 which are arranged on or near the body of the patient 30 to be examined. Such surface coils 15 have a high sensitivity for the reception of magnetic resonance signals which is also spatially inhomogeneous. This means that individual surface coils 15 are mainly sensitive for magnetic resonance signals originating from separate directions, i.e. from separate parts in space of the body of the patient to be examined. The coil sensitivity profile represents the spatial sensitivity of the set of surface coils. The transmission coils, notably surface coils, are connected to a demodulator 24 and the received magnetic resonance signals (MS) are demodulated by means of the demodulator 24. The demodulated magnetic resonance signals (DMS) are applied to a reconstruction unit. The reconstruction unit reconstructs the magnetic resonance image from the demodulated magnetic resonance signals (D)MS) and on the basis of the coil sensitivity profile of the set of surface coils. The coil sensitivity profile has been measured in advance and is stored, for example electronically, in a memory unit which is included in the reconstruction unit. The reconstruction unit derives one or more image signals from the demodulated magnetic resonance signals (DMS), which image signals represent one or more, possibly successive magnetic resonance images. This means that the signal levels of the image signal of such a magnetic resonance image represent the brightness values of the relevant magnetic resonance image. The reconstruction unit 25 in practice is preferably constructed as a digital image processing unit 25 which is programmed so as to reconstruct the magnetic resonance image from the demodulated magnetic resonance signals and on the basis of the coil sensitivity profile. The digital image processing unit 25 is notably programmed so as to execute the reconstruction in conformity with the so-called SENSE technique or the so-called SMASH technique. The image signal from the reconstruction unit is applied to a monitor 26 so that the monitor can display the image information of the magnetic resonance image (images). It is also possible to store the image signal in a buffer unit 27 while awaiting further processing, for example printing in the form of a hard copy.

In order to form a magnetic resonance image or a series of successive magnetic resonance images of the patient to be examined, the body of the patient is exposed to the magnetic field prevailing in the examination space. The steady, uniform magnetic field, i.e. the main field, orients a small excess number of the spins in the body of the patient to be examined in the direction of the main field. This generates a (small) net macroscopic magnetization in the body. These spins are, for example nuclear spins such as of the hydrogen nuclei (protons), but electron spins may also be concerned. The magnetization is locally influenced by application of the gradient fields. For example, the gradient coils 12 apply a selection gradient in order to select a more or less thin slice of the body. Subsequently, the transmission coils apply the RF excitation pulse to the examination space in which the part to be imaged of the patient to be examined is situated. The RF excitation pulse excites the spins in the selected slice, i.e. the net magnetization then performs a precessional motion about the direction of the main field. During this operation those spins are excited which have a Larmor frequency within the frequency band of the RF excitation pulse in the main field. However, it is also very well possible to excite the spins in a part of the body which is much larger man such a thin slice; for example, the spins can be excited in a three-dimensional part which extends substantially in three directions in the body. After the RF excitation, the spins slowly return to their initial state and the macroscopic magnetization returns to its (thermal) state of equilibrium. The relaxing spins then emit magnetic resonance signals. Because of the application of a read-out gradient and a phase encoding gradient, the magnetic resonance signals have a plurality of frequency components which encode the spatial positions in, for example the selected slice. The k-space is scanned by the magnetic resonance signals by application of the read-out gradients and the phase encoding gradients. According to the invention, the application of notably the phase encoding gradients results in the sub-sampling of the k-space, relative to a predetermined spatial resolution of the magnetic resonance image. For example, a number of lines which is too small for the predetermined resolution of the magnetic resonance image, for example only half the number of lines, is scanned in the k-space.

1. Magnetic resonance imaging method for forming, wherein undersampled magnetic resonance signals are acquired by at least one receiver antenna having a plurality of receiver antenna positions, each with a spatial sensitivity profile, the image being reconstructed from the undersampled magnetic resonance signals and the spatial sensitivity profiles, whereas the reconstruction of the image is provided by a first step, in which the image is reconstructed on the basis of reconstruction matrices according to a parallel imaging method, thereinafter the so reconstructed image is subject to a filtering operation, which provides a post-processed image, which is used to alter the reconstruction matrices, and by a second step, in which the final image is reconstructed on the basis of the altered reconstruction matrices:

2. Magnetic resonance method according to claim 1, wherein the filtering is a median filtering method.

3. Magnetic resonance method according to claim 1, wherein the filtering is a wavelet filtering.

4. Magnetic resonance method according to claim 1, wherein the filtering is a low-pass filtering.

5. Magnetic resonance method according to claim 1, wherein the filtering is performed by anisotropic diffusion.

6. Magnetic resonance method according to claim 1, wherein the filtering is performed statistically.

7. Magnetic resonance method according to claim 1, wherein the alteration of the reconstruction matrix is performed based on adjusting the trade-off between the noise level and the artifact level.

8. A magnetic resonance imaging system comprising
a static main magnet having a main magnetic field,
at least one receiver antenna having a plurality of receiver antenna positions,
means for applying a read and other gradients,
means for measuring MR signals along a predetermined trajectory containing a plurality of lines in k-space
a receiver antenna system for acquiring undersampled MR signals, each receiver antenna position having a spatial sensitivity profile,
means for reconstruction of the image in a first step on the basis of recconstruction matrices according to a parallel imaging method,
means for filtering the so reconstructed image, which provides a post-processed image,
means for altering the reconstruction matrices by use of the post-processed images,
means for reconstruction of the final image on the basis of the altered reconstruction matrices.

9. A computer readable media comprising instructions for controlling a computer system to perform a method of forming a magnetic resonance image the method comprising:
applying a read and other gradients,
measuring MR signals along a predetermined trajectory containing a plurality of lines in k-space
acquiring undersampled MR signals from a receiver antenna system, each receiver antenna position having a spatial sensitivity profile,
reconstruction of the image in a first step on the basis of recconstruction matrices according to a parallel imaging method,
filtering the so reconstructed image, which provides a post-processed image,
altering the reconstruction matrices by use of the post-processed images,
reconstruction of the final image on the basis of the altered reconstruction matrices.
